(19) United States
(12) Patent Application Publication
(10) Pub. No.: US 2026/0306165 A1
(43) Pub. Date: Oct. 8, 2026

(54) CATHETERS AND METHODS FOR ENDOVASCULAR TREATMENT WITH ELECTRODES

(71) Applicant: TVA MEDICAL, INC., Franklin Lakes, NJ (US)

(72) Inventors: Oladipo Peter Alerele-Ale, Franklin Lakes, NJ (US); Andrew Moll, Franklin Lakes, NJ (US); Alexander Palmer, Franklin Lakes, NJ (US); Olivia R. Palmer, Franklin Lakes, NJ (US); Kristin N. Rominger, Franklin Lakes, NJ (US); Breanna Simpson, Franklin Lakes, NJ (US)

(21) Appl. No.: 19/100,794

(22) PCT Filed: Aug. 2, 2022
(86) PCT No.: PCT/US2022/039139

§ 371 Date: Feb. 3, 2025

(51) Int. Cl. A61B 18/14; A61B 18/00

(52) U.S. Cl. CPC A61B 18/1492; A61B 18/149; A61B 2018/00184; A61B 2018/00428; A61B 2018/00577; A61B 2018/00601; A61B 2018/1465; A61B 2018/1475

(57) ABSTRACT

Embodiments of catheters and methods for endovascular treatment (e.g., fistula formation) using one or more catheters. Some embodiments of the catheter may include a catheter body including a sidewall, the sidewall defining an opening; and at least two electrodes including ablation surfaces, the at least two electrodes extending through the opening and intersecting one another at one or more locations to define a cutting shape. In other embodiments, the catheter may include a catheter body including a sidewall with an opening formed in the sidewall; and an electrode comprising an ablation surface, the ablation surface having an ablation opening such that the ablation surface splits and joins to define a cutting shape when in a deployed configuration.

### TECHNICAL FIELD

The present specification generally relates to catheters and methods for endovascular treatment of a blood vessel and, more specifically, catheters with electrodes and methods for endovascular treatment of a blood vessel with catheters with electrodes.

### BACKGROUND

Treatments such as endovascular fistula formation may include positioning a catheter with an electrode within concomitant blood vessels to treat the blood vessels. Treatment of the blood vessel may include forming a fistula between two blood vessels. Forming a fistula between two blood vessels can have one or more beneficial functions. For example, the formation of a fistula between an artery and a vein may provide access to the vasculature for hemodialysis patients. Specifically, forming a fistula between an artery and a vein allows blood to flow quickly between the vessels while bypassing the capillaries. In other instances, a fistula may be formed between two veins to form a veno-venous fistula. Depending on the condition of the blood vessels such as a degree of calcification of the walls of the blood vessels, the treatment may require forming a fistula in a certain size or a shape, rather than as a slit. The size and shape of the fistula may depend on the shape of the electrode that ablates or cuts the blood vessels.

Accordingly, a need exists for catheters and methods that provide electrodes with varying sizes or shapes for fistula formation.

### SUMMARY

The present embodiments address the above referenced problems. In particular, the present disclosure is directed to catheters and methods for endovascular treatment of a blood vessel using catheters with electrodes. In embodiments, the presently-disclosed catheters and methods may provide electrodes with varying sizes and shapes for fistula formation and forming the fistulas having different sizes and shapes.

In one embodiment, a catheter for endovascular treatment is provided. The catheter may include a catheter body comprising a sidewall. The sidewall defines an opening. At least two electrodes include ablation surfaces. The at least two electrodes extend through the opening and intersect one another at one or more locations to define a cutting shape.

In another embodiment, a method of forming a catheter is provided. The method may include bending at least two electrodes of the catheter such that the at least two electrodes are shaped to extend away from a centerline of a catheter body of the catheter. The catheter body may include a sidewall defining an opening, the at least two electrodes include ablation surfaces, extend through the opening, and intersect one another at one or more locations to define a cutting shape.

In yet another embodiment, a catheter for endovascular treatment is provided. The catheter may include a catheter body that includes a sidewall with an opening formed in the sidewall, and an electrode comprising an ablation surface. The ablation surface has an ablation opening such that the ablation surface splits and joins to define a cutting shape.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a catheter, according to one or more embodiments shown and described herein;

FIG. 2A schematically depicts a perspective view of a portion of the catheter of FIG. 1, according to one or more embodiments shown and described herein;

FIG. 2B schematically depicts a cross sectional view of a portion of the catheter of FIG. 2A taken along section line 2B-2B, according to one or more embodiments shown and described herein;

FIG. 3A schematically depicts a perspective view of another embodiment of a catheter of FIG. 1, according to one or more embodiments shown and described herein;

FIG. 3B schematically depicts a cross sectional view of a portion of the catheter of FIG. 3A taken along section line 3B-3B, according to one or more embodiments shown and described herein;

FIG. 4A schematically depicts a perspective view of another embodiment of a catheter of FIG. 1, according to one or more embodiments shown and described herein;

FIG. 4B schematically depicts a cross sectional view of a portion of the catheter of FIG. 4A taken along section line 4B-4B, according to one or more embodiments shown and described herein;

FIG. 5A schematically depicts a backstop of another catheter, according to one or more embodiments shown and described herein;

FIG. 5B schematically depicts alignment of the catheter of FIG. 1 and the another catheter of FIG. 5A, according to one or more embodiments shown and described herein; and

FIG. 6 schematically depicts alignment of the catheters of FIG. 1, according to one or more embodiments shown and described herein.

Reference will now be made in greater detail to various embodiments of the present disclosure, some embodiments of which are illustrated in the accompanying drawings. Whenever possible, the same reference numerals will be used throughout the drawings to refer to the same or similar parts.

### DETAILED DESCRIPTION

Embodiments as described herein are directed the systems, methods, and catheters for forming a fistula between two vessels. For example, fistulas may be formed between adjacent arteries and veins, adjacent veins, adjacent arteries, etc. In at least one embodiment, a catheter for endovascular treatment generally includes a catheter body and at least one electrode defining a cutting shape. The electrode may have an opening such that an ablation surface splits and joins to define a cutting shape. The catheter may include at least two electrodes that may intersecting one another at one or more locations to define a cutting shape. As will be described in greater detail, by having the electrode defining a cutting shape, the electrode may provide varying cutting sizes or shapes for forming fistulas having different shapes and sizes other than slits. These and additional embodiments and benefits will be described in greater detail herein.

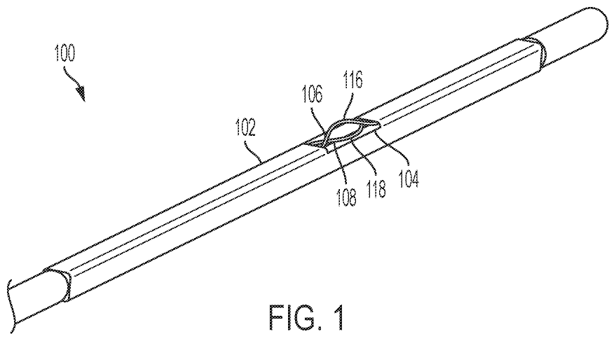

Referring now to FIG. 1, a catheter 100 for endovascular treatment is generally depicted. The catheter 100 may generally include a catheter body 102, electrodes 106 and 108, and an opening 104 in a sidewall of the catheter body 102 through which the electrodes 106 and 108 are extended through. It is noted that the catheter 100 may include a greater or fewer number of components without departing from the scope of the present disclosure.

The catheter body 102 may be sized to be advanced through a blood vessel and may include a distal tip that may be shaped and/or sized to aid in advancement of the catheter 100 through a blood vessel. The catheter body 102 may have any cross-sectional shape and any diameter suitable for intravascular use. The catheter 100 may include or define one or more lumens or other passageways (not shown) extending at least partially along or through the catheter body 102. For instance, the one or more lumens may extend at least partially longitudinally through the catheter body 102 in a longitudinal direction of the catheter 100. The catheter body 102 may be formed of any material or combination of materials able to be traversed through a vasculature of a body.

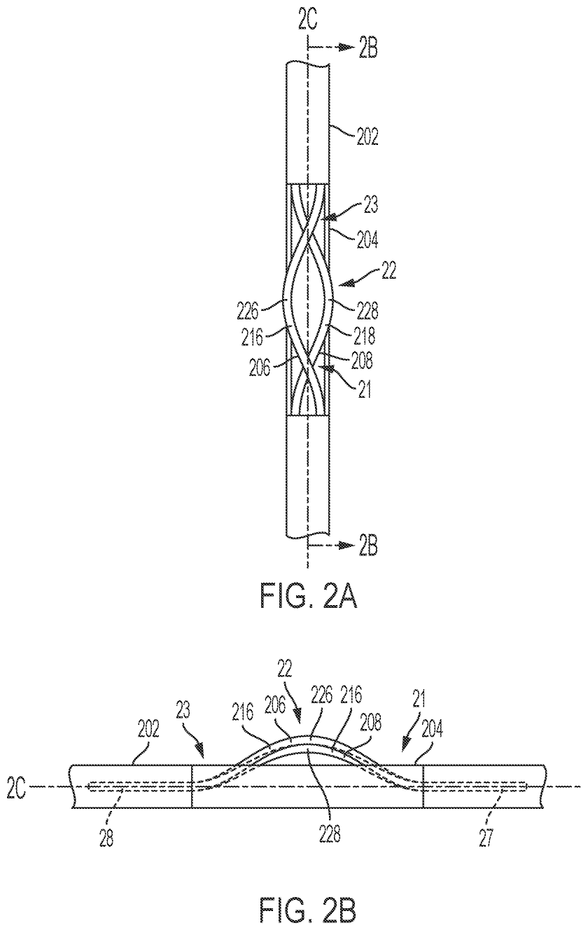

As noted above, the catheter 100 may have the electrodes 106 and 108 for endovascular treatment including cutting a blood vessel or forming a fistula. As depicted, the electrodes 106 and 108 may have ablation surfaces 116 and 118 that may extend through the opening 104. The electrodes 106 and 108 may intersect one another at one or more locations to define a cutting shape. For example, the electrodes 106 and 108 may be crisscrossed with each other in the shape of “X” at two locations, which will be discussed later in detail with reference to FIGS. 2A and 2B. The size and shape ablation surfaces 116 and 118 of the electrodes 106 and 108 may be varied based on factors including tissue thickness and density, degree of calcification in a blood vessel, as well as desired fistula size, shape, and location.

In some embodiments, the electrodes 106 and 108 may be spring wires or leaf spring electrodes, which may be movable between a retracted configuration, in which the electrodes 106 and 108 are retained within the catheter 100, and a protruding configuration, in which the electrodes 106 and 108 project beyond the sidewall of the catheter body 102. The electrodes 106 and 108 may or may not be naturally biased to project from the catheter body 102. When the electrodes 106 and 108 are naturally biased to project from the catheter body 102, a structure, such as a sleeve (not shown), may be used to hold or maintain the electrodes 106 and 108 in a retracted configuration until deployment is desired. In some embodiments, the electrodes 106 and 108 may be manually advanced or retracted such as via a lead wire which may be coupled to the electrodes 106 and 108 that may be manually pulled or pushed to retract or advance the electrodes 106 and 108. The electrodes 106 and 108 may be deployed (e.g., extended through the opening 104) when advanced. In some embodiments, the catheter 100 may comprise one or more insulating materials (not shown) which may shield or otherwise protect the catheter 100 and its components from heat generated by the electrodes 106 and 108 during use. The electrodes 106 and 108 may be coupled to a power source (not shown), such as via a lead wire or other conductor attached thereto. When activated, current may be supplied to and/or carried from tissue and fluid via the ablation surface to facilitate ablation or vaporization of tissue to form a fistula. The catheter 100 may be insulated to protect the catheter 100 and its components from heat generated by the electrodes 106 and 108. For example, one or more portions of the catheter body 102 adjacent to the electrodes 106 and 108 may comprise a heat insulating portion that may be ceramic.

FIGS. 2A and 2B illustrate an embodiment of electrodes intersecting at one or more locations as depicted in FIG. 1. Referring to FIG. 2A, electrodes 206 and 208 may each have an ablation surface 216 and 218 that extends through an opening 204 in a sidewall of a catheter body 202. The ablation surfaces of the electrodes 206 and 208 may intersect one another at two locations to define a cutting shape. For example, the electrodes 206 and 208 may be crisscrossed with each other in the shape of “X” at two locations. The number of locations where the electrodes 206 and 208 intersect one another may be one or more than two to reach desired fistula size or shape. For example, the electrodes 206 and 208 may intersect each other at one location, either at a proximal portion or a distal portion and arced or bent to define a cutting shape similar to the embodiment of FIG. 2A with the two intersecting locations. Additionally, the electrodes 206 and 208 may intersect each other at more than two locations to form a cutting shape. The number of locations where the electrodes 206 and 208 intersect one another may be more than two to reach desired fistula size or shape.

In some embodiments, the electrode 206 may be disposed over the electrode 208 as shown in FIG. 2A. In other embodiments, the electrode 206 may be disposed under the electrode 208. In yet other embodiments, the electrode 206 may be disposed over the electrode 208 at one intersecting location and disposed under the electrode 208 at another intersecting location. Such intertwined configuration of the electrodes 206 and 208 may provide stability to the position of the electrodes 206 and 208. The electrodes 206 and 208 may contact each other at intersecting locations.

In some embodiments, the electrodes 206 and 208 may be arced in a lateral direction that is perpendicular to a long axis of the catheter body 202 as shown in FIG. 2A. The arced electrodes 206 and 208 may, for example, define an oval or almond cutting shape. In some embodiments, the electrodes 206 and 208 may be bent in the lateral direction of the catheter body 202 to form a diamond or trapezoidal-shaped ablation surface. The distance between the electrodes 206 and 208 may be greater than the width of the catheter body 202 as illustrated in FIG. 2A. In this regard, intermediate portions 226 and 228 of the electrodes 206 and 208 may extend laterally outward outside of the catheter body 202. In the example of FIG. 2A, a distance between the electrodes 206 and 208 increases from a first intersecting location 21 to a midpoint location 22 between the first intersecting location 21 and a second intersecting location 23 and then decreases from the midpoint location 22 to the second intersecting location 23. In some embodiments, the distance (e.g., the maximum distance) between the electrodes 206 and 208 may be substantially the same width or smaller than the width of the catheter body 202. The distance between the electrodes 206 and 208 may be designed to provide desired cutting size or shape and/or fistula size or shape. In some embodiments, the distance between the electrodes 206 and 208 may be adjusted by advancing or retracting such as via a lead wire which may be coupled to the electrodes 206 and 208.

Referring to FIG. 2B, a portion of the electrodes 206 and 208 may be housed in the catheter body 202 and a portion of the electrodes 206 and 208 extends through the opening 204. For example, a distal end 25 (not shown) and a proximal end 26 (not shown) of the electrode 206 and a distal end 27 and a proximal end 28 of the electrode 208 may be disposed in the catheter body 202, and the intermediate portion 226 and 228 of each of the electrodes 206 and 208 may be extended through the opening 204. The intermediate portion 226 and 228 of each of the electrodes 206 and 208 may be biased to protrude from the catheter body 202 through the opening 204. In some embodiments, the intermediate portion 226 and 228 of each of the electrodes 206 and 208 may be arced away from the catheter body 202 forming a somewhat concave shape that is arced toward the opening 204. In this regard, the electrodes 206 and 208 may be arced in two directions away from an axially extending centerline 2C of the catheter body 202. In other embodiments, the intermediate portion 226 and 228 of each of the electrodes 206 and 208 may be substantially parallel to the longitudinal direction of the catheter body 202 such that to form an arch shape with a flat top. The shape of the electrodes 206 and 208 may be designed to conform a blood vessel wall or another catheter to form a fistula therebetween. Thickness of the electrodes 206 and 208 or the width of ablation surfaces 216 and 218 of the electrodes 206 and 208 may also be designed to fit the electrodes 206 and 208 in the catheter body 202.

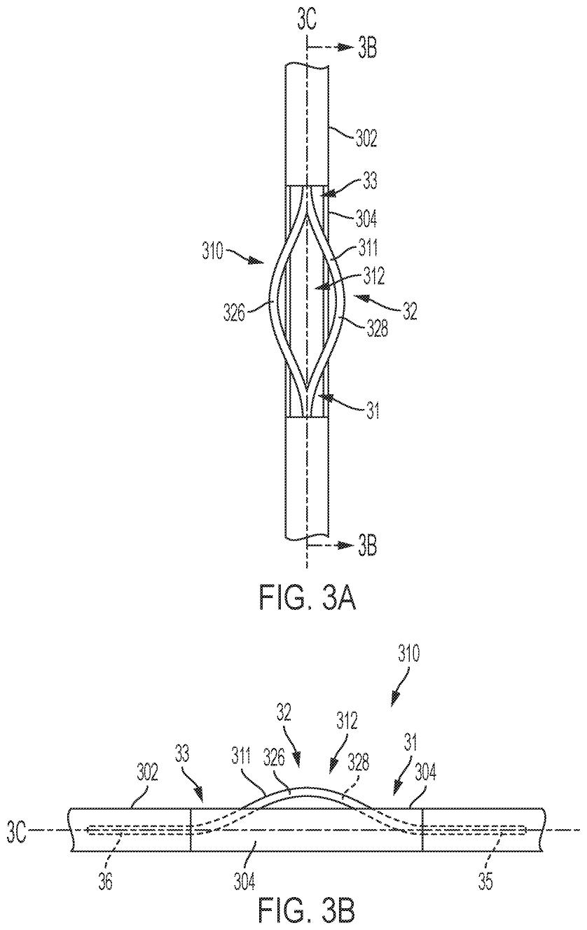

FIGS. 3A and 3B illustrate an embodiment of an electrode 310 having an ablation opening 312. In this embodiment, the electrode 310 is formed as a single monolithic structure rather than of multiple intertwined electrodes. For example, the electrode 310 may have an ablation surface 311 that may split and rejoin to define a cutting shape. Referring to FIG. 3A, the electrode 310 may have a distal portion 31, an intermediate portion 32, and a proximal portion 33. The intermediate portion 32 may include a first intermediate portion 326 and a second intermediate portion 328. For example, the intermediate portion 32 may split at the distal portion 31 of the electrode 310 to the first intermediate portion 326 and the second intermediate portion 328. The first intermediate portion 326 and the second intermediate portion 328 may rejoin at the proximal portion 33 of the electrode 310. The intermediate portion 32 may thus form the ablation opening 312 between the first intermediate portion 306 and the second intermediate portion 308. The ablation opening 312 may define an overall cutting shape of the electrode 310. The general cutting shape of the embodiment illustrated in FIG. 3A is similar to the cutting shape of the embodiment illustrated in FIG. 2A, in which the two electrodes 206 and 208 forming substantially the same outer periphery with that of the electrode 310.

In some embodiments, the first intermediate portion 306 and the second intermediate portion 308 may be arced in a lateral direction that is perpendicular to a long axis of the catheter body 302 as shown in FIG. 3A. For example, the first intermediate portion 306 and the second intermediate portion 308 may define an oval or almond cutting shape. In other embodiments, the first intermediate portion 306 and the second intermediate portion 308 may be bent in a lateral direction of the catheter body 302 to form a diamond or trapezoidal-shaped ablation surface. The distance (e.g., the maximum distance) between the first intermediate portion 306 and the second intermediate portion 308 may be greater than the width of the catheter body 302 as illustrated in FIG. 3A. In this regard, intermediate portions 306 and 308 may extend laterally outward outside of the catheter body 302. In the example of FIG. 3A, a distance between the intermediate portions 326 and 328 increases from a first intersecting location (i.e., the distal portion 31) to a midpoint location (i.e., the intermediate portion 32) between the first intersecting location and a second intersecting location (i.e., the proximal portion 33) and then decreases from the midpoint location (i.e., the intermediate portion 32) to the second intersecting location (i.e., the proximal portion 33). In other embodiments, the distance (e.g., the maximum distance) between the first intermediate portion 306 and the second intermediate portion 308 may be substantially the same with or smaller than the width of the catheter body 202. The distance between the first intermediate portion 306 and the second intermediate portion 308 may be designed to provide desired cutting size or shape and/or fistula size or shape.

Referring to FIG. 3B, the electrode 310 may be partially housed in the catheter body 302 and the intermediate portion 32 of the electrode 310 may extend through the opening 304. For example, a distal end 35 and a proximal end 36 of the electrode 310 may be disposed in the catheter body 302, and the first intermediate portion 326 and the second intermediate portion 328 may be extended through the opening 304. The first intermediate portion 326 and the second intermediate portion 328 may be biased to protrude from the catheter body 302 through the opening 304. In some embodiments, the first intermediate portion 326 and the second intermediate portion 328 may be arced away from the catheter body 302 forming a somewhat concave shape that is arced toward the opening 304. In this regard, the electrode 310 may be arced in two directions away from an axially extending centerline 3C of the catheter body 302. In other embodiments, the first intermediate portion 326 and the second intermediate portion 328 may be substantially parallel to the longitudinal direction of the catheter body 302 and may, for example, form a three centered arch or a pseudo three centered arch shape. The shape of the first intermediate portion 326 and the second intermediate portion 328 may be designed to conform a blood vessel wall or another catheter to form a fistula therebetween. Thickness of the electrode 310 or the width of ablation surface 311 of the electrode 310 may also be designed to fit the electrode 310 in the catheter body 302. In some embodiments, the distal end 35 may be a free end, which may be exposed through the opening 304 when the electrode 310 is deployed.

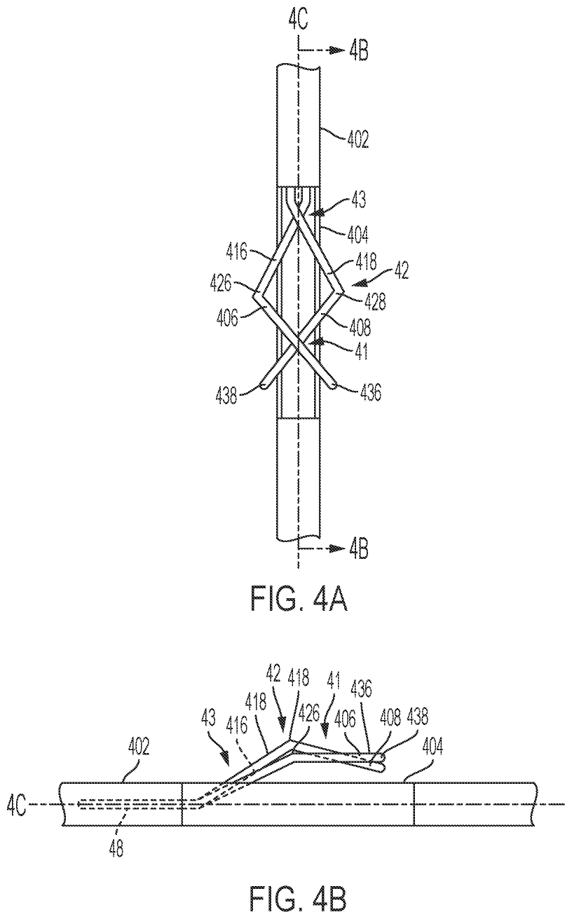

FIGS. 4A and 4B illustrates an embodiment of electrodes intersecting at one or more locations. Referring to FIG. 4A, electrodes 406 and 408 may each have an ablation surface 418 and 416 that extends through an opening 404 in a sidewall of a catheter body 402. The ablation surfaces 416 and 418 of the electrodes 406 and 408 may intersect one another at two locations to define a cutting shape. For example, the electrodes 406 and 408 may be crisscrossed with each other in the shape of “X” at two locations. The number of locations where the electrodes 406 and 408 intersect one another may be one or more than two to reach desired fistula size or shape. For example, the electrodes 406 and 408 may intersect each other at one location, either at a proximal portion or a distal portion and arced or bent to define a cutting shape similar to the embodiment of FIG. 4A with the two intersecting locations. Additionally, the electrodes 406 and 408 may intersect each other at more than two locations to form a cutting shape. The number of locations where the electrodes 406 and 408 intersect one another may be more than two to reach desired fistula size or shape.

In some embodiments, the electrodes 406 may be disposed under the electrode 408 at one location and disposed over the electrode 408 at other location as shown in FIG. 4A. Such intertwined configuration of the electrodes 406 and 408 may provide stability to the position of the electrodes 406 and 408. In other embodiments, the electrode 406 may be disposed under the electrode 408 at both intersecting locations. In yet other embodiments, the electrode 406 may be disposed over the electrode 408 at both intersecting locations. Each of the electrodes 406 and 408 may have a distal end, which may be a free end, and a proximal end, which may be disposed in the catheter body 402. The free ends of the electrodes 406 and 408 may be connected to define a cutting shape. The electrodes 406 and 408 may contact each other at intersecting locations.

In some embodiments, the electrodes 406 and 408 may be arced or bent in a lateral direction that is perpendicular to a long axis of the catheter body 402 as shown in FIG. 4A and may define, for example, an oval, almond, trapezoid or diamond cutting shape. The distance (e.g., the maximum distance) between the electrodes 406 and 408 may be greater than the width of the catheter body 402 as illustrated in FIG. 4A. In this regard, intermediate portions 426 and 428 of the electrodes 406 and 408 may extend laterally outward outside of the catheter body 402. In the example of FIG. 4A, a distance between the electrodes 406 and 408 increases from a first intersecting location 41 to a midpoint location 42 between the first intersecting location 41 and a second intersecting location 43 and then decreases from the midpoint location 42 to the second intersecting location 43. In some embodiments, the distance (e.g., the maximum distance) between the electrodes 406 and 408 may be substantially the same width or smaller than the width of the catheter body 402. The distance (e.g., the maximum distance) between the electrodes 406 and 408 may be designed to provide desired cutting size or shape and/or fistula size or shape.

Referring to FIG. 4B, the electrodes 406 and 408 may be housed in the catheter body 402 and a portion of the electrodes 406 and 408 extends through the opening 404. For example, a proximal end 46 (not shown) of the electrode 406 and a proximal end 48 of the electrode 408 may be disposed in the catheter body 402, and an intermediate portion 426 and 428 and a distal portion 436 and 438 of each of the electrodes 406 and 408 may be extended through the opening 404. The intermediate portion 426 and 428 and the distal portion 436 and 438 of each of the electrodes 206 and 208 may be biased to protrude from the catheter body 402 through the opening 404. In this regard, the electrodes 406 and 408 may be bent or arced in two directions away from an axially extending centerline 4C of the catheter body 402. In other embodiments, the intermediate portion 426 and 428 of each of the electrodes 406 and 408 may be bent or ached such that a plane defined by the distal portions 436 and 438 of the electrodes 406 and 408 may be substantially parallel to the longitudinal direction of the catheter body 402 and may, for example, form a three centered arch or a pseudo three centered arch shape. The shape of the electrodes 406 and 408 may be designed to conform a blood vessel wall or another catheter to form a fistula therebetween. Thickness of the electrodes 406 and 408 or the width of ablation surfaces 416 and 418 of the electrodes 406 and 408 may also be designed to fit the electrodes 406 and 408 in the catheter body 402.

The electrodes discussed herein may be made from any suitable material or combination of materials. In some variations, the electrodes may comprise one or more refractory metals. For example, an electrode may comprise such as but not limited to, tungsten, molybdenum, niobium, tantalum, rhenium, stainless steel, nickel, combinations or alloys thereof including tungsten rhenium, nickel titanium (i.e., nitinol), etc.

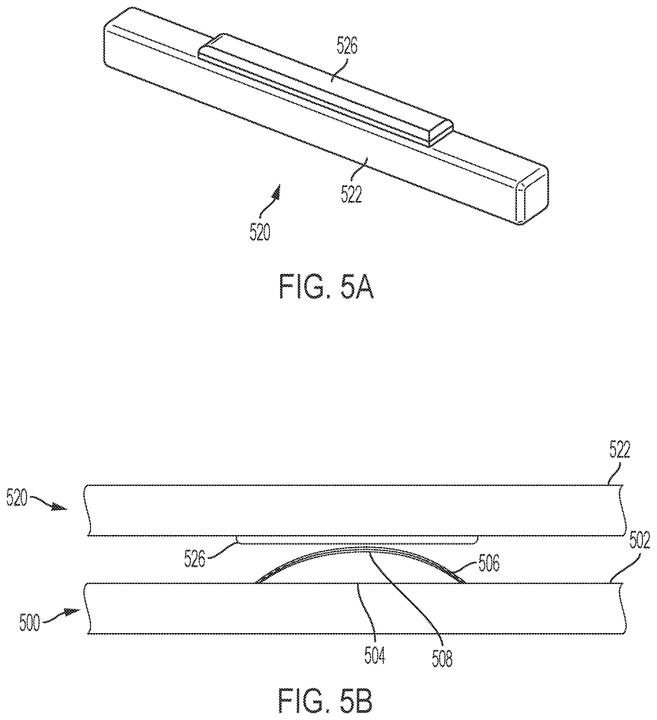

Referring to FIGS. 5A and 5B, in some embodiments, a catheter 520 having a backstop 526 may be used to perform endovascular treatment of a blood vessel in conjunction with any one of the catheters of embodiments discussed hereinabove with reference to FIGS. 1-4B. FIG. 5A illustrates an embodiment of the backstop 526 disposed on the catheter body 522 of the catheter 520. The backstop 526 may be formed from a nonconductive material such as, but not limited to, a ceramic and/or polymeric material, etc. The backstop 526 may be formed from a conductive material as well. In some embodiments, the backstop 526 may be magnetic, formed of a low corrosive metal material (e.g., ferrous or non-ferrous). As depicted, the backstop 526 may be a thin ribbon overlying the catheter body 522. In some embodiments, a top surface of the backstop 526 may have concave or convex portions. FIG. 5B illustrates the catheter 520 and a catheter 500 aligned to perform endovascular treatment on blood vessels (not shown). The catheter 500 generally include a catheter body 502, electrodes 506 and 508, which may be any of the electrodes described herein, and an opening 504 in a sidewall of the catheter body 502.

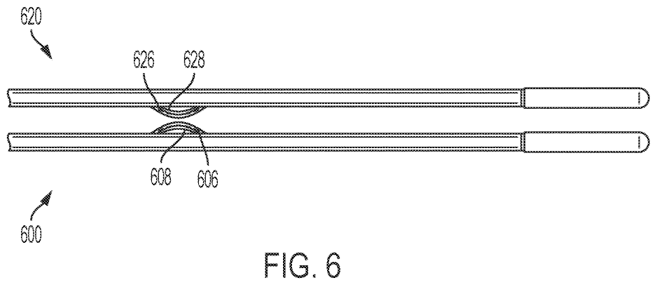

Referring to FIG. 6, in some embodiments, two catheters having at least one electrode may be used to perform endovascular treatment of a blood vessel therebetween. Each of the two catheters may be any one of the catheters of embodiments discussed hereinabove with reference to FIGS. 1-4B. A catheter 600 may include electrodes 606 and 608. A catheter 620 may include electrodes 626 and 628. The catheter 600 and/or the catheter 620 may have a single electrode configuration (e.g., the electrode 310). The catheter 600 and 620 may be aligned to perform endovascular treatment of a blood vessel therebetween.

It is noted that catheters provided herein may include a greater or fewer number of components without departing from the scope of the present disclosure.

As noted herein, devices and methods as provided herein may be used for purposes other than fistula formation. For example, the devices as provided herein may be used for arterializing purposes (e.g., arterializing a vein for ischemia in the leg), vessel occlusion, angioplasty, thrombectomy, atherectomy, crossing, drug coated balloon angioplasty, stenting (uncovered and covered), lytic therapy, etc. In addition, methods provided herein, may include multiple treatments and or multiple treatment sites.

Embodiments can be further described with reference to the following numerical clauses:
1. A catheter for endovascular treatment comprising: a catheter body comprising a sidewall, the sidewall defining an opening; and at least two electrodes comprising ablation surfaces, the at least two electrodes extending through the opening and intersecting one another at one or more locations to define a cutting shape when in a deployed configuration.
2. The catheter of clause 1, wherein the ablation surfaces each define a proximal portion, a distal portion, and an intermediate portion extending between and connecting to the proximal portion and the distal portion, and the one or more locations are located at the intermediate portion of each ablation surface.
3. The catheter of any preceding clause, wherein each of the at least two electrodes comprise a proximal end disposed within the catheter body.
4. The catheter of any preceding clause, wherein each of the at least two electrodes comprise a distal end disposed within the catheter body.
5. The catheter of any preceding clause, wherein each of the at least two electrodes comprise a distal end disposed outside of the catheter body in the deployed configuration.
6. The catheter of any preceding clause, wherein the intermediate portion is biased to protrude from the catheter body to engage a treatment portion of a blood vessel to be treated.
7. The catheter of any preceding clause, wherein the intermediate portion bends in at least two perpendicular directions away from a centerline of the catheter body.
8. A method of forming a catheter comprising at least two electrodes, comprising: bending the at least two electrodes of the catheter such that the at least two electrodes are shaped to extend away from a centerline of a catheter body of the catheter, wherein the catheter body includes a sidewall defining an opening, the at least two electrodes include ablation surfaces, extend through the opening, and intersect one another at one or more locations to define a cutting shape when in a deployed configuration.
9. The method of clause 8, wherein the ablation surfaces each define a proximal portion, a distal portion, and an intermediate portion extending between and connecting to the proximal portion and the distal portion, and the one or more locations are located at the intermediate portion of each ablation surface.
10. The method of any preceding clause, wherein each of at least two electrodes comprise a proximal end disposed within the catheter body.
11. The method of any preceding clause, wherein each of at least two electrodes comprise a distal end disposed within the catheter body.
12. The method of any preceding clause, wherein each of at least two electrodes comprise a distal end disposed outside of the catheter body in the deployed configuration.
13. The catheter of any preceding clause, wherein the intermediate portion of the ablation surface is shaped to be flush with a wall of a treatment portion of a blood vessel to be treated.
14. A catheter for endovascular treatment comprising: a catheter body comprising a sidewall with an opening formed in the sidewall; and an electrode comprising an ablation surface, the ablation surface having an ablation opening such that the ablation surface splits and joins to define a cutting shape when in a deployed configuration.
15. The catheter of clause 14, wherein the ablation surface has a proximal portion, a distal portion, and an intermediate portion extending between and connecting to the proximal portion and the distal portion, and the ablation opening is located at the intermediate portion of the ablation surface.
16. The catheter of any preceding clause, wherein the electrode comprises a proximal end disposed within the catheter body.
17. The catheter of any preceding clause, wherein the electrode comprises a distal end disposed within the catheter body.
18. The catheter of any preceding clause, wherein the electrode comprises a distal end disposed outside of the catheter body in the deployed configuration.
19. The catheter of any preceding clause, wherein the intermediate portion is biased to protrude from the catheter body to engage a treatment portion of a blood vessel to be treated.
20. The catheter of any preceding clause, wherein the intermediate portion is shaped to be flush with a wall of a treatment portion of a blood vessel to be treated.
21. A method for endovascular treatment comprising: advancing a catheter to a treatment portion of a blood vessel, the catheter comprising: a catheter body comprising a sidewall with an opening formed in the sidewall, and one or more electrodes comprising an ablation surface, the ablation surface defining a cutting shape having a hollow center, the one or more electrodes extending through the opening; bending the one or more electrodes such that the one or more electrodes are shaped to extend away from a centerline of the catheter body; and activating the one or more electrodes to perform treatment at the treatment portion.
22. The method of clause 21, wherein the ablation surface defines a proximal portion, a distal portion, and an intermediate portion extending between and connecting to the proximal portion and the distal portion, and the hollow center is located at the intermediate portion of the ablation surface.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A catheter for endovascular treatment comprising:
a catheter body comprising a sidewall, the sidewall defining an opening; and
at least two electrodes comprising ablation surfaces, the at least two electrodes extending through the opening and intersecting one another at one or more locations to define a cutting shape when in a deployed configuration.

2. The catheter of claim 1, wherein the ablation surfaces each define a proximal portion, a distal portion, and an intermediate portion extending between and connecting to the proximal portion and the distal portion, and the one or more locations are located at the intermediate portion of each ablation surface.

3. The catheter of claim 1, wherein each of the at least two electrodes comprise a proximal end disposed within the catheter body.

4. The catheter of claim 1, wherein each of the at least two electrodes comprise a distal end disposed within the catheter body.

5. The catheter of claim 1, wherein each of the at least two electrodes comprise a distal end disposed outside of the catheter body in the deployed configuration.

6. The catheter of claim 2, wherein the intermediate portion is biased to protrude from the catheter body to engage a treatment portion of a blood vessel to be treated.

7. The catheter of claim 2, wherein the intermediate portion bends in at least two perpendicular directions away from a centerline of the catheter body.

8. A method of forming a catheter comprising at least two electrodes, comprising:
bending the at least two electrodes of the catheter such that the at least two electrodes are shaped to extend away from a centerline of a catheter body of the catheter,
wherein the catheter body includes a sidewall defining an opening, the at least two electrodes include ablation surfaces, extend through the opening, and intersect one another at one or more locations to define a cutting shape when in a deployed configuration.

9. The method of claim 8, wherein the ablation surfaces each define a proximal portion, a distal portion, and an intermediate portion extending between and connecting to the proximal portion and the distal portion, and the one or more locations are located at the intermediate portion of each ablation surface.

10. The method of claim 8, wherein each of the at least two electrodes comprise a proximal end disposed within the catheter body.

11. The method of claim 8, wherein each of the at least two electrodes comprise a distal end disposed within the catheter body.

12. The method of claim 8, wherein each of the at least two electrodes comprise a distal end disposed outside of the catheter body in the deployed configuration.

13. The catheter of claim 9, wherein the intermediate portion of the ablation surface is shaped to be flush with a wall of a treatment portion of a blood vessel to be treated.

14. A catheter for endovascular treatment comprising:
a catheter body comprising a sidewall with an opening formed in the sidewall; and
an electrode comprising an ablation surface, the ablation surface having an ablation opening such that the ablation surface splits and joins to define a cutting shape when in a deployed configuration.

15. The catheter of claim 14, wherein the ablation surface has a proximal portion, a distal portion, and an intermediate portion extending between and connecting to the proximal portion and the distal portion, and the ablation opening is located at the intermediate portion of the ablation surface.

16. The catheter of claim 14, wherein the electrode comprises a proximal end disposed within the catheter body.

17. The catheter of claim 14, wherein the electrode comprises a distal end disposed within the catheter body.

18. The catheter of claim 14, wherein the electrode comprises a distal end disposed outside of the catheter body in the deployed configuration.

19. The catheter of claim 15, wherein the intermediate portion is biased to protrude from the catheter body to engage a treatment portion of a blood vessel to be treated.

20. The catheter of claim 15, wherein the intermediate portion is shaped to be flush with a wall of a treatment portion of a blood vessel to be treated.
